(19) United States
(12) Patent Application Publication
(10) Pub. No.: US 2012/0088960 A1
(43) Pub. Date: Apr. 12, 2012

(54) BOWEL PACKING DEVICE HAVING A TETHER

(76) Inventors: Steven J. Kubisen, Ellicott City, MD (US); Anthony P. Deasey, Severna Park, MD (US); Dennis Smith, Ellicott City, MD (US)

(21) Appl. No.: 13/166,634

(22) Filed: Jun. 22, 2011

Related U.S. Application Data
(60) Provisional application No. 61/392,462, filed on Oct. 12, 2010.

(51) Int. Cl. A61F 2/04

(52) U.S. Cl. 600/37

(57) ABSTRACT

A kit for packing the bowels of a subject. The kit comprises an elastomeric bowel packing device manually positioned within the subject to retain the bowels of the subject in an operational displaced position and to provide a surgical operational space; and an elongate tether having a distal end coupled to the device via an attachment arrangement.

### CROSS REFERENCE TO RELATED APPLICATIONS

The contents of U.S. Provisional Patent Application No. 61/392,462 filed on Oct. 12, 2010, U.S. Provisional Patent Application 61/125,219 filed on Apr. 23, 2008, and PCT/US2009/002495 filed on Apr. 22, 2009, are hereby incorporated herein by reference in their entirety.

### BACKGROUND

1. Field of the Invention

The present invention relates generally to bowel packing, and more particularly, to a bowel packing device having a tether.

2. Related Art

Abdominal and pelvic procedures generally require displacement and retention of bowels or other organs to create a space that allows the surgeon to perform the procedure. This step of displacement and retention of bowels is referred to herein as bowel packing.

The current packing procedure used in the operating room today is time-consuming relative to the overall priorities of events in a surgery. The surgeon first uses his hands to displace the bowels away from the surgical site. Intra-abdominal sponges and towels are then used to pack the bowels out of the way. Finally, abdominal retractors are fitted over the dressings with gentle traction to hold the cotton sponges in place.

This conventional bowel packing causes several issues during surgery. For instance, bowel packing may take up to ten minutes, and, because the bowels have a tendency to protrude from the dressing into the surgical space, the bowel packing must be repeated frequently during extended surgical procedures. Additionally, the cotton sponges used to pack the bowels are made of loose cotton fibers that can adhere to the bowels, and remain within the subject even after removal of the sponges. These fibers can promote peritoneal inflammation, a major cause of post-operative adhesion formation. Furthermore, the sponges tend to dry out over the course of the surgical procedure, becoming abrasive and adhesive to the bowels themselves, further contributing to the formation of adhesions, a leading cause of post-operative complications and morbidity. Finally, because multiple sponges are used, there is a danger that one or more sponges will be forgotten in the abdominal cavity.

### SUMMARY

In one aspect of the present invention, a kit for packing the bowels of a subject is provided. The kit comprises: an elastomeric bowel packing device manually positioned within the subject to retain the bowels of the subject in an operational displaced position and to provide a surgical operational space; and an elongate tether having a distal end configured to be coupled to the device via an attachment arrangement.

In another aspect of the present invention, a method of packing bowels of a subject with an elastomeric bowel packing device is provided. The method comprises: attaching, via an attachment arrangement, a tether to the device; and positioning, through a surgical incision, the device and tether in the subject such that the tether drapes through the incision.

### BRIEF DESCRIPTION OF THE DRAWINGS

Embodiments of the present invention are described herein in conjunction with the accompanying drawings, in which:

FIG. 1A is a front view of a bowel packing device, in accordance with embodiments of the present invention;

FIG. 1B is a side view of a bowel packing device, in accordance with embodiments of the present invention;

FIG. 2A is a back view of a bowel packing device, in accordance with embodiments of the present invention;

FIG. 2B is cross-sectional view of an attachment arrangement, in accordance with embodiments of the present invention;

FIG. 2C is side view of an attachment arrangement, in accordance with embodiments of the present invention;

FIG. 2D is cross-sectional view of an attachment arrangement, in accordance with embodiments of the present invention;

FIG. 2E is cross-sectional view of an attachment arrangement, in accordance with embodiments of the present invention;

FIG. 2F is cross-sectional view of an attachment arrangement, in accordance with embodiments of the present invention;

FIG. 2G is cross-sectional view of an attachment arrangement, in accordance with embodiments of the present invention;

FIG. 3A is a top view of a bowel packing device, in accordance with embodiments of the present invention;

FIG. 3B is a perspective view of a tether that may be used in embodiments of the present invention;

FIG. 3C is a perspective view of a tether that may be used in embodiments of the present invention; and

FIG. 4 is a flowchart illustrating a method for attaching a tether to a bowel packing device body, in accordance with embodiments of the present invention.

### DETAILED DESCRIPTION

Aspects of the present invention are generally directed to a device for packing the bowels of a subject. The device comprises an elastomeric body adapted to be operationally positioned within the subject. The device comprises an elongate tether that has a distal end coupled to the body via an attachment arrangement.

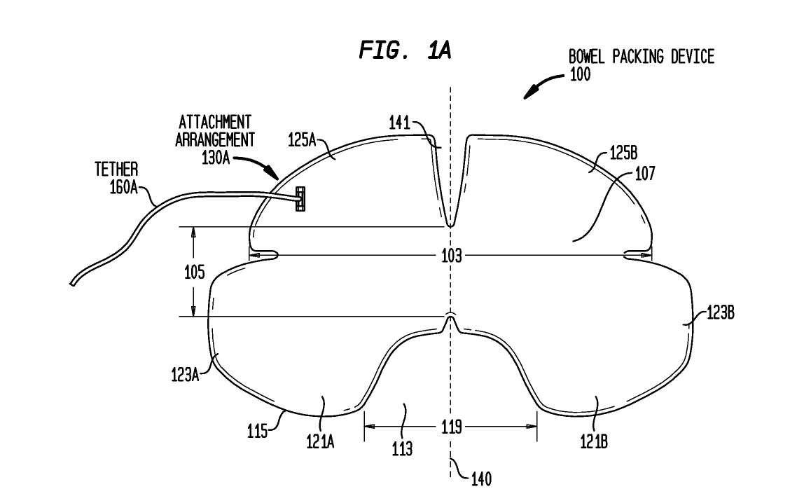

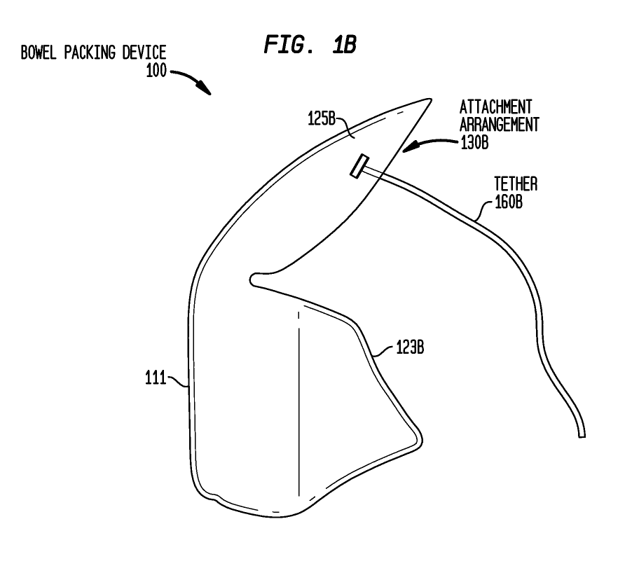

FIG. 1A is a front view of a bowel packing device 100 in accordance with embodiments of the present invention. As shown, bowel packing device 100 is made from an elastomeric material and has a tether 160A. Device 100 comprises, in this embodiment, an essentially rectangular central portion 107 having a width 103 and a height 105. Device 100 further comprises first and second top flaps 125 separated by a notch 141, and first and second bottom flaps 121 separated by a notch 113. Additionally, body 107 further comprises two side flaps 123.

Central portion 107 and flaps 121, 123, and 125, collectively form an essentially elliptical shape. More specifically, device 100 has an essentially elliptically-shape, that is generally symmetrical about a minor axis 140 of the device. In certain embodiments, flaps 121, 123 and 125, and central portion 107 of the device are one contiguous structure, preferably made from a single piece of elastomeric material or compound such as a silicon polymer.

Notch 113 in FIG. 1A is a bell-shaped cut with in the bottom edge 115 of device 100, and is provided to accommodate the ventral medial part of the subject's body in the sagittal plane, and designed to conform to, and provide space for, the spinal cord towards the ventral wall of the abdominal cavity).

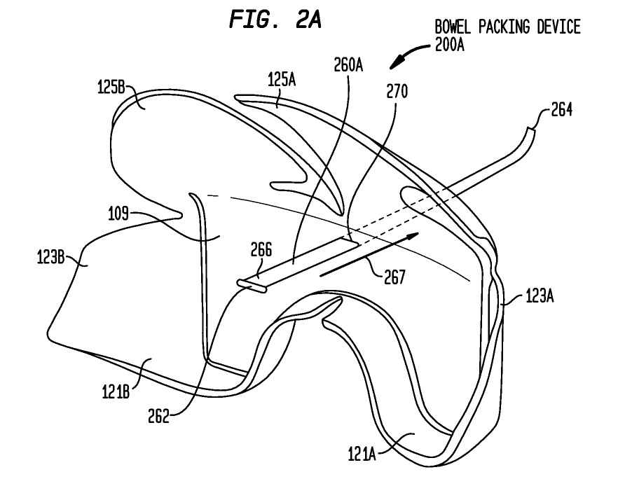

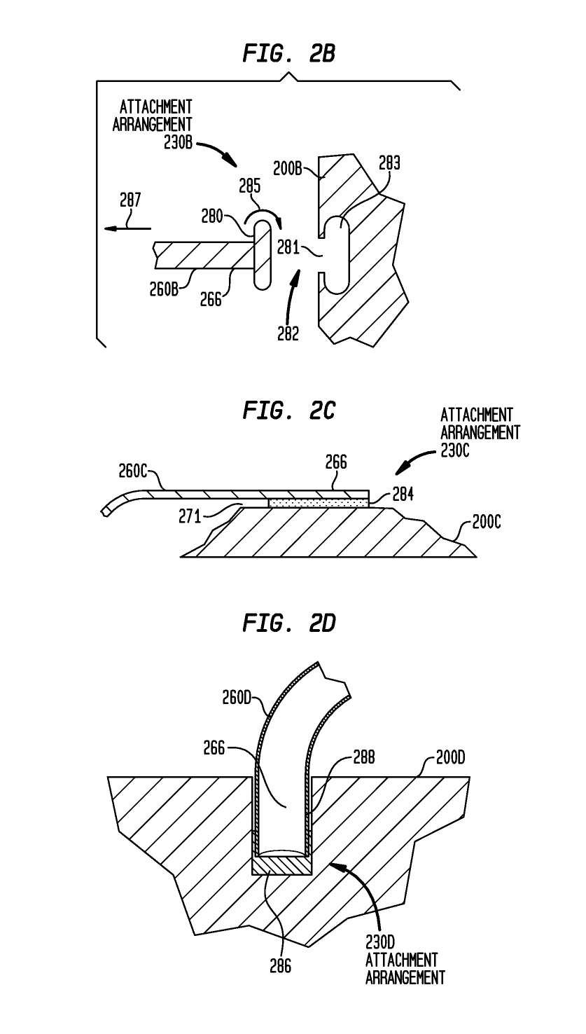

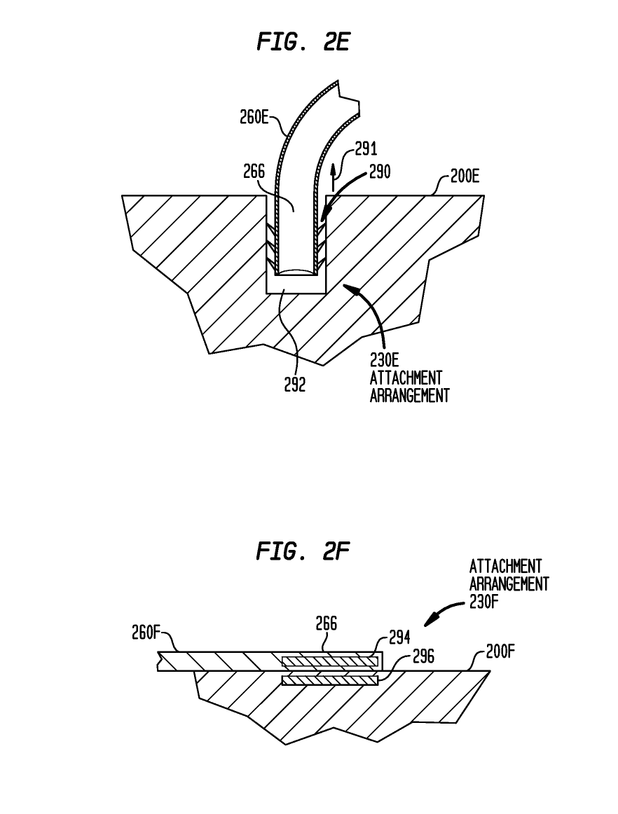

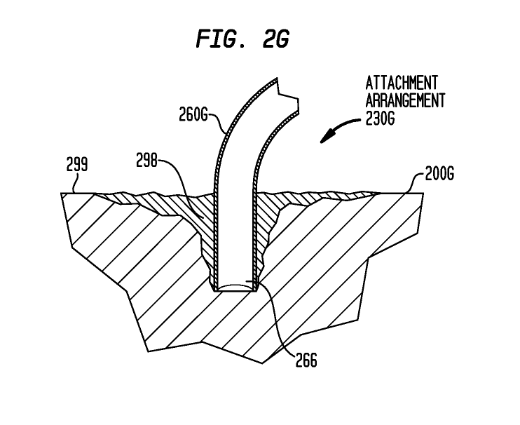

As previously noted, during a bowel packing procedure, a surgeon displaces the subject's bowels to create a space that allows the surgeon to perform the procedure. Device 100 is used to retain the bowels in this displaced position, thereby providing a barrier that maintains the surgical space. More specifically, a first surface 109 (FIG. 2A) of device 100 abuts the subject's bowel, and a second surface 111 interfaces with retractor Hades. As used herein, bowels generically include bowel, intestine, and other abdominal organs that would need to be displaced in the abdominal cavity to allow for abdominal surgery. The standard refractor blade setup uses two blades that interface with the lateral sides of the body. Additionally, upon insertion of device 100 into a subject, side flaps 123 contact the lateral sides of the abdominal cavity and top flaps 125 contact the ventral side of the abdominal cavity. Side flaps 123 serve to aid in containing bowels that may protrude around the sides of the device in the abdominal cavity. The purpose of top flaps 123 is to help secure the bowels on the ventral side of the subject. In other words, device 100 is dimensioned to cover the bowels of the subject when operationally positioned within the abdomen of the subject.

In certain embodiments, bowel packing device 100 is appropriately sized for bowel packing of a subject. That is, the device is dimensioned to allow for insertion into the abdominal cavity of the subject. For example, in adult humans, the size of the abdominal cavity is about 3.9 to 5.8 inches in the transverse plane at the height of the base of the ribs and about 7.6 to 11.3 inches in the coronal plane at the height of the base of the ribs. An appropriately sized device for bowel packing in a mammal having such dimensions is about 5.2 to about 7.5 inches overall height (from ventral to dorsal sides of the abdominal cavity upon placement) and about 8.7 to about 12.5 inches in overall width (from lateral side to lateral side of the abdominal cavity upon placement). However, it would be appreciated that device 100 may have different sizes and shapes, depending on, for example, the insertion technique, surgical procedure, subject, etc. In certain embodiments, portion 107 has a width 103 that is approximately 7.82 inches, and height 105 that is approximately 3.63 inches. In such embodiments, notch 112 has a height 117 of approximately 2.28 inches and a base width 119 of approximately 4.00 inches. Further details of the body of a bowel packing device in accordance with the embodiments of FIG. 1A are described in International Patent Application No. PCT/US2009/002495, filed on Apr. 22, 2009, the contents of which are hereby incorporated by reference herein.

As previously noted, in the embodiments of FIG. 1A, device 100 further comprises a tether 160A. Tether 160A is attached to top flap 125A of the device via an attachment arrangement 130A. Different types of attachment arrangements, and different locations for such attachment arrangements, are described further below. In certain embodiments of the present invention, tether 160A functions as an indicator that bowel packing device 100 remains in the subject, while in the same or different embodiments, tether 160A provides a means for the surgeon to remove device 100 from the subject.

In embodiments of the present invention, tether 160 is formed from a non-fibrous material. For example, in certain embodiments, tether 160 is an elastomeric material that is similar to the material used for device 100, as described elsewhere herein. In other embodiments, tether 160 is formed from materials generally available to a surgeon during a surgical procedure, such as suture materials, a catheter, etc.

FIG. 1B is a side view of device 100 of FIG. 1A and illustrates one exemplary alternative location for an attachment arrangement 130B and tether 160B. More specifically, in the embodiments of FIG. 1B, an attachment arrangement 130B is located in top flap 125B, which is on the opposing side of axis 140 from flap 125A.

FIG. 2A is a back view of another variation of a bowel packing device, referred to as device 200A, in accordance with embodiments of the present invention. In these embodiments, bowel packing device 200A is substantially similar to bowel packing device 100 of FIGS. 1A and 1B, including main portion 107 and flaps 121, 123 and 125.

FIG. 2A illustrates one exemplary attachment arrangement 230A for attaching tether 260A to bowel packing device 200A. Attachment arrangement 230A comprises an aperture 270 positioned in device 200A, and a stop 262 positioned at distal end 266 of tether 260A. Stop 262 has a configuration (size and/or shape), such that it will not pass through aperture 270. Therefore, in use, proximal end 264 of tether 260A is inserted through aperture 270, and pulled in the direction shown by arrow 267 until the stop abuts aperture 270. Because stop 262 abuts aperture 270 and will not pass there through, device 200A can be manipulated through application of tensile forces to tether 260A without detachment of tether 260A from device 200A.

In embodiments of the present invention, manipulation of device 200A may include removing the body from its operational position abutting the subject's bowels. That is, in such embodiments the strength of the attachment provided by the interface between stop 262 and aperture 270 is sufficient such that device 200A can be removed from the subject by the surgeon pulling on the tether.

FIG. 2A illustrates an embodiment in which device 200A includes one aperture that is configured to receive proximal end 264 of tether 260A. In other embodiments the device may include a plurality of apertures to receive proximal end 264 of tether 260A.

As noted, FIG. 2A illustrates embodiments in which tether 260A includes a stop 262 that interfaces with aperture 270. As described below, in other embodiments, stop is not included and tether 260A is formed from a material that allows the surgeon to use aperture 270 to tie distal end 266 around a portion of the device.

FIG. 2B is a cross-sectional view of a section of a bowel packing device 200B, and illustrates another attachment arrangement 230B that may be implemented in embodiments of the present invention. Attachment arrangement 230B includes a slot 282 in the body of device 200B, and a clip 280 disposed at the distal end 266 of tether 260B. Clip 280 and slot 282 have corresponding shapes such that when clip 280 is inserted into slot 282, the clip is securely retained therein.

More specifically, in the embodiments of FIG. 2B, slot 282 has an elongate portion 283, that is approximately the same shape as elongate tubular clip 280. However, portion 283 is larger in size than clip 280. Slot 282 further comprises an opening 281 that is smaller than elongate portion 283, but is large enough that clip 282, if properly rotated and/or aligned, can be inserted there through. In operation, clip 282 is titled as shown by arrow 285, and inserted through opening 281 into elongate portion 283. During, or following insertion into elongate portion 283, clip 282 is permitted to rotate so as to be coaxially aligned with elongate portion 283. In such an aligned position, when a tensile force is placed on tether 260B, clip 282 will abut the surfaces of elongate portion 283 adjacent to opening 281, but will be retained within elongate portion. As such, because clip 280 abuts opening 281, but will not pass there through, device 200B can be manipulated through application of tensile forces to tether 260B without detachment of tether 260B from device 200B.

In embodiments of the present invention, manipulation of device 200B may include removing the device from its operational position abutting the subject's bowels. That is, in such embodiments the strength of the attachment provided by the interface between clip 280 and slot 282 is sufficient such that the device can be removed from the subject by the surgeon pulling on tether 260B.

FIG. 2B illustrates embodiments of the present invention in which clip 280 and slot 282 both have generally elongate, tubular shapes. In other embodiments, clip 280 is a spherical element, and slot 282 has a corresponding spherical shape.

FIG. 2B illustrates one slot 282 positioned in device 200B. It would be appreciated that in alternative embodiments device 200B may include a plurality of slots to receive clip 280. Therefore, in such embodiments, a surgeon may select the location at which tether 260B should be attached to device 200B.

FIG. 2C is a side view of a section of a bowel packing device 200C, and illustrates another attachment arrangement 230C that may be implemented in embodiments of the present invention. In this embodiment, attachment arrangement 230C is an adhesive 284 disposed between the distal end 266 of tether 266C and the surface 271 of bowel packing device 200C. Adhesive 284 may comprise, for example silicone adhesives, RTV silicone adhesives, cyanoacrylate adhesives, polyurethane adhesives, epoxy adhesives and other adhesive capable of bonding the tether material to the bowel packing device material. Adhesives that are formulated to remain flexible (vs. brittle) when cured are preferred. (such as silicone adhesives, latex adhesives, and polyurethane adhesives). The use of adhesive 284 as an attachment arrangement allows the surgeon to attach tether 266C to device 200C at a number of different locations, making the device highly customizable for the needs of the recipient.

In certain embodiments of FIG. 2C, adhesive 284 forms a bond between tether 266C and device 200C that has sufficient strength so as to enable manipulation of device 200C through application of tensile forces to tether 266C. Therefore, when sufficient when a tensile force is applied to tether 266C to manipulate device 200C, the adhesive bond will not break.

In embodiments of the present invention, manipulation of device 200C may include removing the device from its operational position abutting the subject's bowels. That is, in such embodiments the strength of the attachment provided by adhesive 284 is sufficient such that device 200C can be removed from the subject by the surgeon pulling on tether 266C.

FIG. 2D is a cross-sectional view of a section of a bowel packing device 200D, and illustrates another attachment arrangement 230D that may be implemented in embodiments of the present invention. In this embodiment, attachment arrangement 230D is a recess 288 in device 200D, and an adhesive 286 disposed around distal end 266 of tether 266D within recess 288. Similar to the above embodiments of FIG. 2D, adhesive 286 forms an adhesive bond between tether 266D and device 200D such that, when a tensile force is applied to tether 266D, the adhesive bond will not break, thereby permitting manipulation of device 200D via the tether without detachment of tether 260D from device 200D. Adhesive 284 may comprise, for example silicone adhesives, RTV silicone adhesives, cyanoacrylate adhesives, polyurethane adhesives, epoxy adhesives and other adhesive capable of bonding the tether material to the bowel packing device material. Adhesives that are formulated to remain flexible (vs. brittle) when cured are preferred. (such as silicone adhesives, latex adhesives, and polyurethane adhesives).

FIG. 2E is a cross-sectional view of a section of a bowel packing device 200E, and illustrates another attachment arrangement 230E that may be implemented in embodiments of the present invention. In this embodiment, attachment arrangement 230E is a receptacle 292 in device 200E, and a connector 290 configured to mechanical mate with the receptacle.

In embodiments of the present invention, receptacle 292 and connector 290 may have a number of different arrangements that allow mechanical coupling of tether 260E to device 200E. FIG. 2E illustrates one specific embodiment in which receptacle 292 is a tubular shaped recess, and connector 290 is a plurality of barbs positioned at the distal end 266 of tether 260E. As shown, barbs 290 are oriented such that the barbs do not significantly interfere with the insertion of distal end 266 into recess 292. However, barbs 290 the orientation of barbs 290 are such that, when a tensile force is placed on tether 260E that pulls distal end 266 out of recess 292 (shown by arrow 291), barbs 290 will engage the outer surface of recess 290. This engagement retains distal end 266 within the recess and maintains attachment between tether 260E and device 200E. Therefore, device 200E may be manipulated through application of tensile forces to tether 260E without detachment of tether 260E from device 200E.

In embodiments of the present invention, manipulation of device 200E may include removing the device from its operational position abutting the subject's bowels. That is, in such embodiments the strength of the attachment provided by barbs 290 and recess 290 is sufficient such that device 200E can be removed from the subject by the surgeon pulling on tether 266E.

FIG. 2F is a cross-sectional view of a section of a bowel packing device 200F, and illustrates another attachment arrangement 230F that may be implemented in embodiments of the present invention. In this embodiment, attachment arrangement 230F comprises a magnet 294 positioned at distal end 266 of tether 260F and a magnetic element 296 at the surface of device 200F. It would be appreciated that magnet 294 and magnetic element 296 may be disposed in, or positioned on, distal end 266 and device 200F, respectively.

Magnetic element 296, is a magnet or a piece of ferromagnetic material that, when magnet 294 is brought in proximity there to, produces a magnetic coupling with the magnet. The strength of the magnetic coupling is sufficient such that device 200F may be manipulated through tensile forces applied to tether 260F without detachment of tether 260F from device 200F. In embodiments of the present invention, manipulation of device 200F may include removing the device from its operational position abutting the subject's bowels. That is, in such embodiments the strength of the attachment provided by magnet 294 and magnetic element 296 is sufficient such that device 200F can be removed from the subject by the surgeon pulling on tether 266F.

In the embodiments of FIG. 2F, magnet 294 is embedded in distal end 266 of tether 260F, and a piece of magnetic element 296 is embedded in device 200F. As noted, magnetic element 296 may be a magnet or a piece of ferromagnetic material. It alternative embodiments of the present invention, magnetic element 296 is a magnet, and a piece of ferromagnetic material is disposed at distal end 266 of tether 260F.

FIG. 2G is a cross-sectional view of a section of a bowel packing device 200G, and illustrates another attachment arrangement 230G that may be implemented in embodiments of the present invention. In this embodiment, attachment arrangement 230G is a solvent that is applied to surface 299 of device 200G to temporarily softens device 200G. In use, distal end 266 of tether 260G is inserted into the softened portion, shown in FIG. 2G as section 298, and section 298 will harden around distal end 266. When section 298 hardens, tether 260G is securely attached to device 200G such that device 200G may be manipulated through tensile forces applied to tether 260G without detachment of tether 260G from device 200G. In embodiments of the present invention, manipulation of device 200G may include removing the device from its operational position abutting the subject's bowels. That is, in such embodiments the strength of the attachment hardened section 298 is sufficient such that device 200G can be removed from the subject by the surgeon pulling on tether 266G.

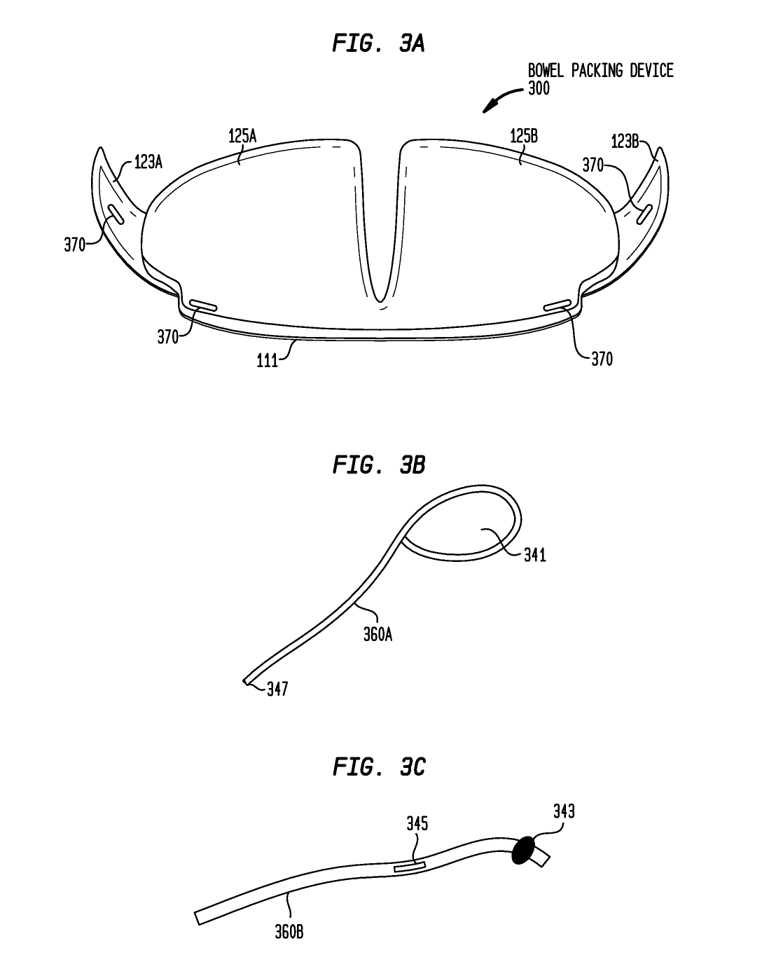

FIG. 3 is top view of an alternative bowel packing device, referred to as device 300, having a plurality of apertures 370 for mating with a tether, in accordance with embodiments of the present invention. In one such embodiment, apertures 370 may be configured to mate with a stop, as described with reference to FIG. 2A.

In another embodiment of FIG. 3A, apertures 370 provide locations for a surgeon to tie or otherwise secure the distal end of a tether to device 300. In one such embodiment, the surgeon forms a tether from a length of material that is available in the operating room, such as catheter tubing or suture material, and ties the end of the material to device 300 using apertures 370. In other embodiments, as described below with reference ton FIGS. 3B and 3C, the tether has a specific arrangement that facilitates attachment to device 300.

More particularly, in the embodiments of FIG. 3B, a tether 360A has a loop 341 formed into the distal end thereof. In operation, the surgeon inserts proximal end 347 through an aperture 370, and then forms a larger loop by pulling the proximal end through loop 341. The surgeon continues to pull proximal end 347 until the larger loop collapses, and tether 360A is pulled tightly against device 300.

In the embodiments of FIG. 3C, tether 360B has a button 343 at the distal end thereof, and a slot 345 formed therein. In operation, the surgeon inserts the distal end and button 343 through an aperture 370, and then brings the button through slot 345. Slot 345 and button 343 are sized and/or shaped such that once the slot secures the button.

In embodiments of the present in which multiple apertures to locations for attachment of the tether are provided, a surgeon may select which of the plurality of apertures to mate with the tether, allowing the attachment location to be customized to the needs or desires of the surgeon. This may be useful due to the face that surgeons use different methods that may require different tether attachment points.

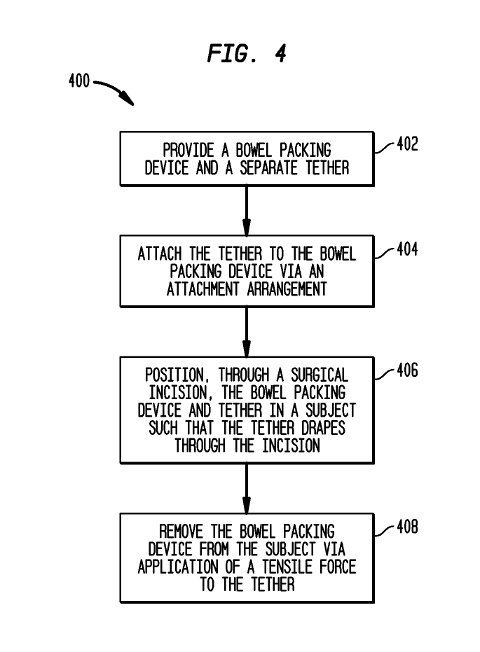

FIG. 4 is a flowchart of a method 400 of using a bowel packing device in accordance with embodiments of the present invention. Method 400 begins at step 402 where a bowel packing device and a separate tether are provided. At step 402, the tether is attached to the bowel packing device via an attachment arrangement described above. At step 406, the bowel packing device and attached tether are positioned within a subject such that the tether drapes through the incision. In certain embodiments, this step may include gaining access to the cavity through, for example, a laparoscopic procedure or a more invasive procedure, such as a laparotomy. During this step, the surgeon repositions the bowels to provide a surgical space in the abdominal cavity of the subject. This may be done by hand, in the case of a laparotomy, or remotely using a probe or the like in the case of a laparoscopic procedure. Next, the device is positioned abutting the bowels of the subject.

After step 406, the surgeon performs the surgical procedure and at block 408 the bowel packing device is removed from the subject. In specific embodiments, the device is removed through application of a tensile force to the tether.

Aspects of the present invention have been primarily described with reference to attachment arrangements that allow the surgeon to attach the tether to the body of the device before or during surgery. Such arrangements are referred to herein as surgical attachment arrangements.

In other embodiments of the present invention, the attachment arrangement is formed during manufacture of the device. In one such embodiment, the distal end of the tether is molded into the device during manufacture, and the attachment arrangement is the portion of the device that surrounds and retains the distal end of the tether. Such embodiments are referred to herein as integral attachment arrangements.

Additionally, FIGS. 2A-3 illustrate specific attachment arrangements that may be implemented in embodiments of the present invention. It would be appreciated that these embodiments are merely illustrative, and are not limiting to the type of attachment arrangements that may be used in embodiments of the present invention. For example, hook and loop fasteners, alternative clips, a sliding turning or pressure locking mechanisms, etc., may all be used in embodiments of the present invention. Furthermore, in certain embodiments, the main portion of the tether is rotatable relative to the attachment arrangement.

As noted above, a bowel packing device may be formed from an elastomeric material or compound such as a silicon polymer. As used herein, “elastomeric compound” is understood as an elastic compound having an appropriate flexibility/rigidity, tear resistance, and sterilization resistance for use in the devices of the invention. Elastomeric compounds for use for manufacture of the device of the invention are sufficiently flexible to prevent damage from occurring to tissues or organs by contact with the device when in a non-compressed state. Elastomeric compound as used herein typically refers to an elastomeric polymer. The monomers that link to form the polymer are typically made from of carbon, hydrogen, oxygen and/or silicon. Examples of elastomeric polymers include Liquid Silicone Rubbers (LSR) and Silicone Encapsulants. In a preferred embodiment of the invention, the elastomeric polymer is a “silicone polymer”. A “silicone polymer” is understood as any silicone-based polymeric material that has the appropriate flexibility/rigidity, tear resistance, and sterilization resistance for use in the devices of the invention. In a preferred embodiment, the silicone polymer is optically clear. Elastomeric compounds for use in the device of the invention include, but are not limited to silicone, liquid silicone rubber (LSR), polydimethylsiloxane (PDMS), styrene butadiene rubber, styrene butadiene styrere (SBS) rubber, nitrile rubber, and polychloroprene (Neoprene). In one embodiment, silicone polymer is polydimethylsiloxane (PDMS) a silicone-based organic polymer. PDMS is optically clear, and is generally considered to be inert, non-toxic and non-flammable. It is occasionally called dimethicone and is one of several types of silicone oil (polymerized siloxane).

A tether in accordance with embodiments of the present invention may also be made from the same or different elastomeric compound as the device body. However, tether may also be made from different materials such as, for example, a non-fibrous cloth, flexible tubing, nylon/polyethylene/polyvinylchloride/silicone/polyurethane/other flexible polymer webbing, suture material, etc.

Bowel packing devices in accordance with embodiments of the present invention are preferably made in different sizes for use in subjects of different sizes (e.g., children and adults). Further, it is understood that the device can be made for use in non-human subjects, for example in pets and other domesticated and non-domesticated animals of value to humans, for example cats, dogs, non-human primates, animals used for medical research including surgical research such as pigs, zoo animals, etc. The disclosure provides information regarding the preferred sizes of the device for use in adult humans, and the size of the abdominal cavity of adult humans. Provided with this information, a device can be made that is appropriately sized for use in mammals other than human adults. Such modifications are well within the ability of those of skill in the art.

The packing devices and/or tethers of the invention can also include other components such as coatings to reduce sticking of the device to the bowel by coating with polymers, particularly biocompatible polymers, of with commercially available coatings such as Seprafilm®. The coatings may be drug eluting. The coatings may be applied by bulk application, molecular conjugation with the body material, or through nanostructure formation. Examples of possible coatings include: SEPRAFILM®, INTERCEED®, SIROLIMUS®, PACLITAXEL®, EVEROLIMUS®, TRANILAST®, DACRON®, SPRAYGEL®, ADHffilT®, TEFLON®, PRECLUDE® Gore, SyntheMed REPEL-CV®, DuraGen, ADCON'M P (Gliatech), REPEUM and RESOLVE™ (Life Medical Sciences), INTERGEL™ (formerly LUBRICOAT®), icodextrin, hyaluronic acid, heparin, dextran, tissue plasminogen activator, corticosteroids, non-steroid inflammatory drugs (NSAIDS) such as ibuprophen, chondroitin sulfate, carboxymethylcellulose, dexamethosane, tissue plasminogen including recombinant tissue plasminogen, oxyphenbutazone, collagen, collagen inhibitors, polylactic acid, polyglycolic acid, alginic acid, polycaprolactone, glycosaminoglycans, polyethylene oxide (PEO), polyethylene oxidepolypropylene oxide copolymer in any monomeric ratio (pEG-PPO-PEG), hydroxy ethyl methyl acrylate (HEMA), poly(N-isopropylacrylamide) (NIPAAm), polytetraflouroethylene (PTFE), polyesters, and silane, or modification by radio frequency gas discharge (RFGD), and radiation grafting, polytetrafluoroethylene (PTFE), polylactic acid, polyglycolic acid, alginic acid, polycaprolactone, glycosaminoglycans, HEMA, ePTFE, polyesters, carboxymethylcellulose, dexamethasone, tissue plasminogen including recombinant tissue plasminogen, oxyphenbutazone, corticosteriods, icodextrin, hyaluronic acid, hyaluronan, and collagen inhibitors.

Alternatively, packing devices and/or tethers can be coated with agents, for example, anti-microbial agents such as anti-viral agents or anti-bacterial agents. The use of such agents may be useful for the protection of the subject as well as the surgical staff and to reduce the possibility of transmission of infection from subjects infected with HIV, hepatitis, especially drug-resistant forms of hepatitis, methicillin resistant staphylococcus aureus (MERSA), etc.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All documents, patents, journal articles and other materials cited in the present application are hereby incorporated by reference.

Reference herein to “one embodiment” or “an embodiment” means that a particular feature, structure, operation, or other characteristic described in connection with the embodiment may be included in at least one implementation of the invention. However, the appearance of the phrase “in one embodiment” or “in an embodiment” in various places in the specification does not necessarily refer to the same embodiment. It is further envisioned that a skilled person could use any or all of the above embodiments in any compatible combination or permutation.

What is claimed is:

1. A kit for packing the bowels of a subject comprising:
an elastomeric bowel packing device manually positioned within the subject to retain the bowels of the subject in an operational displaced position and to provide a surgical operational space; and
an elongate tether having a distal end configured to be coupled to the device via an attachment arrangement.

2. The kit of claim 1, wherein the bowel packing device has an essentially elliptical shape that is generally symmetrical about a minor axis of the device.

3. The kit of claim 1, wherein the attachment arrangement comprises:
a clip positioned at the distal end of the tether; and
a slot in the device configured to receive and securely retain the clip therein.

4. The kit of claim 3, wherein the clip is an elongate element having its longitudinal axis positioned substantially orthogonal to a longitudinal axis of the tether.

5. The kit of claim 3, wherein the clip is a spherical element, and the slot is a spherical shaped slot.

6. The kit of claim 3, wherein the device includes a plurality of slots configured to receive the clip.

7. The kit of claim 1, wherein the attachment arrangement comprises:
an aperture extending through the device; and
a stop at the distal end of the tether having a configuration that prevents passage of the stop through the aperture,
wherein the tether is insertable from its proximal end into the aperture so that the stop is positioned abutting the aperture.

8. The kit of claim 7, wherein the device includes a plurality of apertures configured to receive the proximal end of the tether.

9. The kit of claim 1, wherein the attachment arrangement comprises:
an adhesive disposed between the distal end of the tether and the device.

10. The kit of claim 1, wherein the attachment arrangement comprises:
a solvent that temporarily softens the device such that when the tether is inserted into the softened portion, the device hardens around the tether.

11. The kit of claim 1, wherein the attachment arrangement comprises:
a recess; and
one or more barbs disposed at the distal end of the tether, wherein the barbs retain the distal end of the tether within the recess following insertion therein.

12. The kit of claim 1, wherein the attachment arrangement comprises:
a magnetic coupling arrangement having a first magnetic element at the surface of the device and a second magnetic element at the distal end of the tether.

13. The kit of claim 1, wherein the tether is molded to the device, and wherein the attachment arrangement comprises a device portion surrounding the tether that is formed during molding.

14. The kit of claim 1, wherein the tether is composed of a material that is different than the elastomer material of the device.

15. The kit of claim 14, wherein the tether is a non-fibrous material.

16. The kit of claim 1, wherein the tether is an elastomeric compound.

17. The kit of claim 1, further comprising:
a plurality of flaps attached to the device; and
a plurality of notches located in the periphery of the device, wherein each of the plurality of notches separate two of the plurality of flaps.

18. The kit of claim 1, wherein an element of the attachment arrangement is disposed in at least one of the plurality of flaps.

19. The kit of claim 1, wherein the attachment arrangement comprises:
an aperture extending through the device, and wherein the distal end of the tether extends through the aperture and is tied around a portion of the device.

20. The kit of claim 1, wherein the attachment arrangement has a strength that prevents detachment of the tether from the device when a tensile force is applied to the tether that is sufficient to will dislodge the device from its operational position within the subject

21. A method of packing bowels of a subject with an elastomeric bowel packing device, the method comprising:
attaching, via an attachment arrangement, a tether to the device; and
positioning, through a surgical incision, the device and tether in the subject such that the tether drapes through the incision.

22. The method of claim 21, further comprising:
removing the device from the subject through application of a tensile force to the tether.

23. The method of claim 21, wherein the attachment arrangement comprises a clip positioned at the distal end of the tether, and a slot in the body, and wherein attaching the tether to the body comprises:
inserting the clip into the slot such that the clip is securely retained therein.

24. The method of claim 21, wherein the attachment arrangement comprises an aperture extending through the body, and a stop at the distal end of the tether that is larger than the aperture, and wherein attaching the tether to the body comprises:
inserting the proximal end of the tether into the aperture so that the stop is positioned abutting the aperture.

25. The method of claim 21, wherein the attachment arrangement comprises an adhesive, and wherein attaching the tether to the body comprises:
placing the adhesive on the surface of the body; and
placing the distal end of the tether abutting the adhesive.

26. The method of claim 21, wherein the attachment arrangement comprises a solvent, and wherein attaching the tether to the body comprises:
placing the solvent on the surface of the body to soften a portion of the body;
placing the distal end of the tether into the softened portion of the body; and
allowing the solvent to dry so that the body hardens around the distal end of the tether.

27. The method of claim 21, wherein the attachment arrangement comprises a recess, and one or more barbs disposed at the distal end of the tether, and wherein attaching the tether to the body comprises:
inserting the distal end of the tether into the recess such that the barbs engage the body and retain the distal end of the tether within the recess.

28. The method of claim 21, wherein the attachment arrangement comprises a magnetic coupling arrangement having a first magnetic element at the surface of the body and a second magnetic element at the distal end of the tether, and wherein attaching the tether to the body comprises:
placing the first and second magnetic elements in proximity to one another to form a magnetic coupling.

29. The method of claim 21, wherein the attachment arrangement comprises an aperture extending through the body, and wherein attaching the tether to the body comprises:
tying the distal end of the tether around a portion of the body.
